(19) United States
(12) Patent Application Publication
(10) Pub. No.: US 2019/0382670 A1
(43) Pub. Date: Dec. 19, 2019

(54) A PROCESS FOR SELECTIVELY REMOVING DIOLEFINS FROM A GAS STREAM

(71) Applicant: HALDOR TOPSØE A/S, Kgs. Lyngby (DK)

(72) Inventors: Karin MADSEN, Søborg (DK); Henrik Junge MORTENSEN, Taastrup (DK); Jens Michael POULSEN, Frederikssund (DK); Mads Kristian KAARSHOLM, Birkerød (DK)

(73) Assignee: HALDOR TOPSØE A/S, Kgs. Lyngby (DK)

(21) Appl. No.: 16/465,636

(22) PCT Filed: Nov. 22, 2017
(86) PCT No.: PCT/EP2017/080072

§ 371 Date: May 31, 2019

(30) Foreign Application Priority Data
Dec. 6, 2016 (DK) PA 2016 00745

(51) Int. Cl. C10G 67/06; B01J 23/28; B01J 21/04; C07C 7/12; C07C 7/13; C07C 7/163

(52) U.S. Cl. CPC C10G 67/06; B01J 23/28; B01J 21/04; C07C 7/12; C07C 7/13; C07C 7/163; C10G 2300/1092; C10G 2300/207; C10G 2300/70; C07C 2523/28; C07C 2521/04; C10G 2400/20; C10G 2300/1088; C10G 2300/202

(57) ABSTRACT

In a process for hydrotreatment of a gas stream containing both olefins and diolefins as well as organic sulfur compounds, the gas stream is introduced into a pre-treatment reactor, where diolefins are reacted with hydrogen in the presence of a supported Mo-catalyst not containing Co or Ni, whereby the diolefins are substantially converted to olefins. Then the gas stream is introduced into a hydrotreater reactor having a higher inlet temperature than the pre-treatment reactor, in which the gas stream is reacted with hydrogen in the presence of a hydrotreating catalyst under hydrodesulfurisation process conditions, whereby the olefins are substantially converted to paraffins and the organic sulfur compounds are converted to H₂S, which is removed by subjecting the hydrotreated gas to a chemisorption or physisorption treatment.

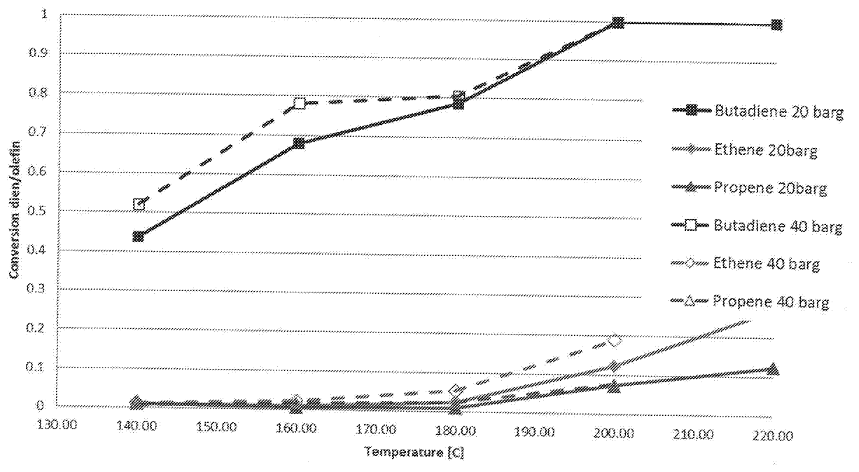

The present invention relates to a process for selective removal of diolefins from a gas stream, preferably from a fuel gas stream, which contains both diolefins and monoolefins, prior to hydrodesulfurisation in a hydrotreatment reactor. The selective removal of diolefins is carried out in a pre-treatment reactor located upstream the hydrotreatment reactor, in which organic sulfur compounds are converted to H₂S.

Diolefins can react to form polymers when a fuel gas stream containing them is passing through a hydrotreatment preheater and reactor. This diolefin reaction may cause severe fouling in the gas hydrotreatment preheater and reactor, and therefore it is necessary to remove the diolefins as completely as possible before they enter the hydrotreatment preheater and reactor.

In the refining industry, sulfur removal or recovery is a very important issue. Sulfur is among the most dominant contaminants in petroleum fractions, and legislation not only limits the permissible sulfur content of finished products, but also limits refinery emissions to the atmosphere. Furthermore, there is a tendency towards the imposition of even more stringent sulfur requirements for fuel gas streams. Therefore, sulfur removal and recovery is a vital process for refineries and gas plant operations. In most locations, the sulfur is hydrotreated and thus converted to hydrogen sulfide, which can be scrubbed from the various liquid or gas streams. The hydrogen sulfide collected from the hydrotreaters and/or gas plants can subsequently be treated, e.g. by the Claus process.

As regards prior art in the field, U.S. Pat. No. 8,921,630 describes a process for the removal of sulfur from a fuel gas stream that, in addition to organic sulfur compounds, contains diolefins and oxygen. The fuel gas stream is treated in a pre-treatment reactor in order to significantly reduce the amounts of any diolefins and oxygen contained therein prior to the hydrodesulfurisation in a hydrotreatment reactor wherein organic sulfur compounds are converted to H₂S. More specifically, in the pre-treatment reactor the fuel gas stream is brought into contact with hydrogen in the presence of a catalyst comprising a group VIb metal, such as Mo, and a group VIII metal selected from Co and Ni on a porous refractory oxide support under mild hydrotreating conditions. These conditions include a temperature of 150 to 350° F. (66 to 177° C.), a pressure in the range of 50 to 400 psig and a gaseous hourly space velocity in the range from 0.05 to 4000 hr¹.

A number of prior art documents specifically mention supported catalysts comprising a group VIb metal and a group VIII metal as being well-suited for hydrotreating purposes. Thus, U.S. Pat. No. 5,507,940 discloses a catalyst in which a liquid form of a silicon compound is incorporated into an alumina-supported group VIb and group VIII metal based catalyst and calcined in an oxidizing atmosphere. U.S. Pat. No. 7,557,062 and U.S. Pat. No. 7,749,937 also disclose hydrotreating catalysts based on a group VIb and group VIII metal based catalyst and a refractory oxide material, which comprises more than 50 wt % titania.

WO 2014/087364 discloses a hydrotreating catalyst especially suited for preparing diesel-range hydrocarbons from a feed containing vegetable oils, comprising a group VIb and a group VIII metal impregnated on a non-refractory oxide as support.

WO 2009/026090 discloses a process for removing sulfur from a refinery fuel gas stream that additionally contains from 2 ppmv to 2.0 wt % diolefins and oxygen as well as organic sulfur compounds. The fuel gas stream is pre-treated in a pre-treatment reactor in order to significantly reduce the amounts of any diolefins and oxygen contained therein prior to the hydrodesulfurization in a hydrotreater reactor wherein organic sulfur compounds are converted to hydrogen sulfide. The hydrogen sulfide formed is removed from the hydrotreated gas stream by use of an absorption treatment method, such as amine treatment, to yield a treated fuel gas stream having a reduced concentration of hydrogen sulfide and an overall sulfur content that is low enough to meet stringent sulfur regulation requirements.

U.S. Pat. No. 6,686,309 describes a catalyst for selective hydrogenation of unsaturated diolefinic compounds, which can also eliminate mercaptans. The catalyst comprises a particulate support selected from alumina, silica, silica-alumina, magnesia or mixtures thereof, palladium and at least one metal selected from molybdenum and tungsten.

Other supported catalysts of this type are disclosed in WO 2014/033653, U.S. Pat. Nos. 7,968,069, 6,306,289, 7,557,062 and US 2013/0153467.

Catalysts based on a group VIb metal only, i.e. not containing a group VIII metal, are not known specifically for use in connection with gas hydrotreatment processes. They are, however, well known for use within other process fields. Thus, WO 02/32570 describes Mo-catalysts supported on bimodal alumina, which are useful for hydrodemetallation of heavy hydrocarbons. As sulfur components are converted into H₂S during a hydrotreatment process, metals will be deposited onto the catalyst as metal sulfides, which will poison or occlude catalytic metal sites that are predominantly located in the catalyst pores, leading to rapid deactivation of the catalyst.

Applicant's EP 2 334 757 B1 describes a process for the production of a hydrocarbon fuel from a renewable organic material by hydrodeoxygenation (HDO). The HDO catalyst is an unpromoted supported Mo-catalyst with a Mo content of 0.1 to 20 wt %, which does not comprise Co and Ni. The support is selected from alumina, silica, titania and combinations thereof, and it has a bimodal porous structure with pores having a diameter as measured by mercury intrusion porosimetry larger than 50 nm, that constitute at least 2 vol % of the total pore volume. By using a carrier with a bimodal pore distribution, the catalyst is more resistant to pore plugging and minimizes increases in pressure drop and deactivation rate.

It has now surprisingly been found that a supported Mo-containing catalyst as described in EP 2 334 757 B1, that does not contain Ni or Co, i.e. a group VIb metal without a group VIII metal, can effectively convert diolefins to olefins at a temperature between 140° C. and 180° C., a pressure in the range of 20 to 45 barg and a gaseous hourly space velocity up to 22500 NL/kg/h.

More specifically, it has been found that a Mo-only catalyst, such as the one developed for fixed-bed hydrodeoxy-genation (HDO) service, effectively converts diolefins into olefins with only negligible olefin conversion in the temperature window T=140-180° C.

This is relevant for fuel gas streams, which contain high levels of olefins, because of the highly exothermic nature of the olefin hydrogenation. If a substantial olefin conversion takes place over the pre-treatment reactor having high levels of olefins (i.e. >4 vol %), a considerable temperature rise may take place, reaching temperatures where diolefins start to polymerize. Furthermore, since the olefin conversion is minimized and primarily diolefin hydrogenation takes place, the exotherm over the pre-treatment reactor can be controlled meticulously. This enables designing the system layout with just a simple low-cost fired heater.

The main technical novelty of this approach lies in a modification of the pre-treater catalyst to selectively treat diolefins rather than monoolefins in order to provide appropriate temperatures in the hydrotreatment reactor in a cost-effective way.

Thus the present invention relates to a process for hydrotreatment of a gas stream containing both olefins and diolefins as well as organic sulfur compounds, said process comprising:

introducing the gas stream into a pre-treatment reactor, where diolefins are reacted with hydrogen in the presence of a supported Mo-catalyst not containing Co or Ni at a temperature of 140-180° C., a pressure of 3-45 barg and a gaseous hourly space velocity up to 22500 NL/kg/h, whereby the diolefins are substantially converted to olefins,

introducing the gas stream, now depleted in diolefins, into a hydrotreater reactor having a higher inlet temperature than the pre-treatment reactor, in which the fuel gas stream is contacted with hydrogen in the presence of a hydrotreating catalyst under hydrodesulfurisation process conditions, whereby the olefins are substantially converted to paraffins and the organic sulfur compounds are converted to H₂S, and

subjecting the hydrotreated gas to a chemisorption or physisorption treatment to remove the H₂S.

The gas stream is preferably a fuel gas stream.

It is preferred that the gas stream has a diolefin content between 2 ppmv and 2 vol %, and it is further preferred that the gas stream contains olefins up to a level of 20 vol %.

It is especially preferred that the gas stream has an olefin content of 1-15 vol %.

The support is preferably selected from alumina, silica, titania and combinations thereof.

The supported Mo-catalyst preferably has a Mo content of 0.1 to 20 wt %.

The gaseous hourly space velocity in the pretreatment reactor preferably is between 500 and 10000 NL/kg/h, most preferably between 1000 and 7000 NL/kg/h.

The invention is illustrated further by the example which follows.

### EXAMPLE

A simulated fuel gas containing 325 ppmv 1,3-butadiene, 1.3% ethane and 1.3% propene was passed over a catalyst bed of an alumina-supported catalyst containing Mo, but not containing Ni or Co, at a gaseous hourly space velocity of 22472 Nl/kg/h.

The detailed gas composition is given in Table 1 below.

TABLE 1
Detailed gas composition

| component | concentration | |
|---|---|---|
| H₂ | 14.9 | vol % |
| C₂H₄ | 1.3 | vol % |
| C₃H₆ | 1.3 | vol % |
| CO | 0.8 | vol % |
| CO₂ | 0.6 | vol % |
| H₂S | 1.0 | vol % |
| N₂ | 79.5 | vol % |
| H₂O | 0.7 | vol % |
| 1,3-butadiene | 325 | ppmv |

The operating conditions are given in Table 2 below.

TABLE 2
Operating conditions

| | |
|---|---|
| Pressure | 20-40 barg |
| Temperature | 140-220° C. |
| Catalyst load | 4.45 g |
| Flow | 100 Nl/h |

The measured conversions of 1.3-butadiene and olefins (ethene and propene) at a pressure of 20/40 barg for a temperature of 140-220° C. are shown in the figure. The butadiene is primarily converted to the corresponding olefin. It was found that the selectivity of the conversion of butadiene to butenes was above 85% at all conditions tested.

1. A process for hydrotreatment of a gas stream containing both olefins and diolefins as well as organic sulfur compounds, said process comprising:
introducing the gas stream into a pre-treatment reactor, where diolefins are reacted with hydrogen in the presence of a supported Mo-catalyst not containing Co or Ni at a temperature of 140-180° C., a pressure of 3-45 barg and a gaseous hourly space velocity up to 22500 NL/kg/h, whereby the diolefins are substantially converted to olefins,
introducing the gas stream, now depleted in diolefins, into a hydrotreater reactor having a higher inlet temperature than the pre-treatment reactor, in which the gas stream is reacted with hydrogen in the presence of a hydrotreating catalyst under hydrodesulfurisation process conditions, whereby the olefins are substantially converted to paraffins and the organic sulfur compounds are converted to H₂S, and
subjecting the hydrotreated gas to a chemisorption or physisorption treatment to remove the H₂S.

2. Process according to claim 1, wherein the gas stream is a fuel gas stream.

3. Process according to claim 1, wherein the gas stream has a diolefin content between 2 ppmv and 2 vol %.

4. Process according to claim 1, wherein the supported Mo-catalyst has a Mo content of 0.1 to 20 wt %.

5. Process according to claim 1, wherein the catalyst support is selected from alumina, silica, titania and combinations thereof.

6. Process according to claim 1, wherein the gas stream contains olefins up to a level of 20 vol %.

7. Process according to claim 6, wherein the gas stream has an olefin content of 1-15 vol %.

8. Process according to claim 1, wherein the gaseous hourly space velocity in the pretreatment reactor is between 500 and 10000 NL/kg/h.

9. Process according to claim 8, wherein the gaseous hourly space velocity in the pretreatment reactor is between 1000 and 7000 NL/kg/h.
